(19) United States
(12) Patent Application Publication
(10) Pub. No.: US 2009/0259301 A1
(43) Pub. Date: Oct. 15, 2009

(54) Detector for abnormal conditions inside body

(76) Inventors: Daniel Gelbart, Vancouver (CA); Lindsay Stuart Machan, Vancouver (CA)

Correspondence Address: DANIEL GELBART, 4706 DRUMMOND DR, VANCOUVER, BC, V6T 1B4, CA

(21) Appl. No.: 12/081,048

(22) Filed: Apr. 10, 2008

(51) Int. Cl. A61F 2/06; A61M 37/00

(52) U.S. Cl. 623/1.42; 604/891.1

(57) ABSTRACT

An implant capable of sensing pressure, force, pH level or any other condition releases a chemical into the body when a pre-set limit is exceeded. The chemical is chosen to be detectable by the person without the need of any external equipment. Typical chemicals are dyes causing coloration visible to the person or chemicals causing a mild and clearly identified reaction such as odor, taste, or unique sensation. The released chemical can have therapeutic effects as well. The invention is particularly suitable for detection of abnormal increase of internal pressure in order to monitor stent grafts.

### FIELD OF THE INVENTION

The invention is in the medical field and in particular in the field of endovascular stent grafts.

### BACKGROUND OF THE INVENTION

In many medical fields it is desired to monitor a condition inside the body and alarm the patient when an abnormal situation develops. Prior art is mainly based on two technologies: electronic modules, being able to transmit a message to the outside of the body or non-electronic devices being periodically monitored by radiological means such as x-ray, MRI or CT. In both cases the detection of the abnormal condition relies on external equipment, requiring the patient to follow a schedule of periodic tests. An example of a common condition requiring monitoring is an aneurysm in an artery, typically the aorta, repaired by a stent graft. A common malfunction of such grafts is a leakage. Any leakage from the repaired area will cause internal bleeding. This can be detected by monitoring the pressure in the area surrounding the graft. Normal pressure in this area will be between 50 mmHg to 80 mmHg, well below systolic blood pressure. Should a leak develop, pressure in the aneurism area outside the graft will exceed 100 mmHg, as it is driven by the blood pressure inside the artery. An example of non-electronic prior art solutions is disclosed in U.S. Pat. No. 6,669,647 and 20060074479. These patents disclose sensors that can be read from outside the body by an X-Ray machine or other radiographic methods. They require the patient to undergo periodic radiography. It is an object of the invention to provide a warning to the patient without the use of any external equipment and without the patient being required to take any action to detect the warning. This will greatly shorten the duration of the period the abnormal condition exists. It is another object to provide a device with practically infinite lifetime. A further object is to provide a simple, low cost device that can be delivered percutaneously. These and further advantages of the invention will become clear from the disclosure and drawings.

### SUMMARY OF THE INVENTION

An implant capable of sensing pressure, force, pH level or any other condition releases a chemical into the body when a pre-set limit is exceeded. The chemical is chosen to be detectable by the person without the need of any external equipment. Typical chemicals are dyes causing coloration visible to the person or chemicals causing a mild and clearly identified reaction such as odor, taste, or unique sensation. The released chemical can have therapeutic effects as well. The invention is particularly suitable for detection of abnormal increase of internal pressure in order to monitor stent grafts.

### BRIEF DESCRIPTION OF THE DRAWINGS

FIG. 1 is a longitudinal cross section of an aneurism repaired by a stent graft being monitored by a device according to the invention.

FIG. 2 is a perspective view of a pressure sensitive device according to the invention.

FIG. 3A is a cross section of a stent graft incorporating a device according to the invention under normal conditions.

FIG. 3B is a cross section of a stent graft incorporating a device according to the invention under abnormal conditions.

FIG. 4 is a cross section of a device capable of detecting a bending force or a temperature change.

### DETAILED DESCRIPTION

Many abnormal conditions inside the body can turn dangerous in a short time. Current methods of periodic testing have to trade off cost of monitoring with risk of delayed detection. For example, when an endovascular graft is installed to prevent an aneurysm from bursting, patients are typically monitored annually at a great cost to the medical system. Should bleeding develop detection time can be as long as twelve month with an average detection time of six months. The invention allows near-instant detection without the costs of periodic monitoring. In order to eliminate the need of external detection equipment and to provide practically infinite life and low cost, a chemical is released inside the body to alarm the patient when a preset limit was exceeded. The alarm mechanism takes advantage of the senses of the patient to detect the chemical, as explained later on.

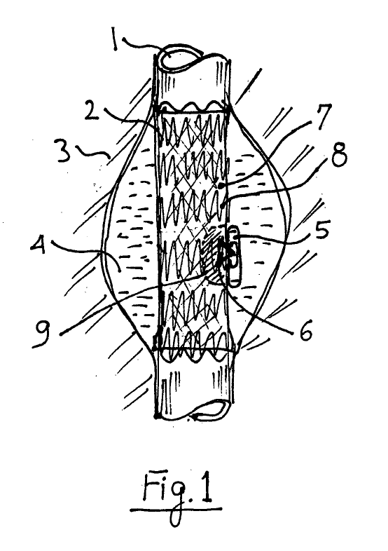

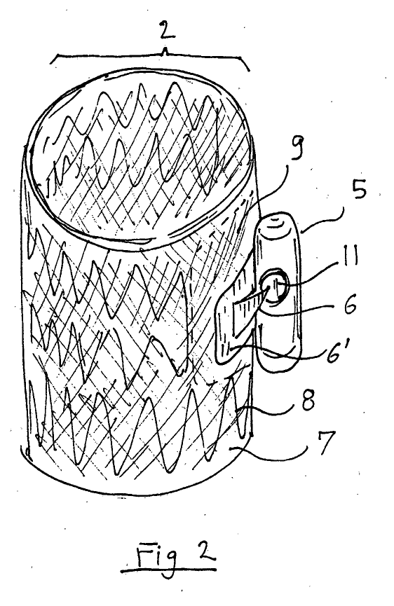

An illustrative example of the invention is a detector for warning the patient against internal bleeding caused by a defect in an aneurysm repair by stent graft. Referring now to FIG. 1 and FIG. 2, an artery 1, surrounded by tissue 3, developed an aneurism 4 that was repaired by stent graft 2 or any other method. A detector 5 is attached to the stent graft 2 and deployed at the same time as the graft. Detector 5 comprises of a sealed container containing a chemical and at least one sharp spike capable of piercing a thin metal foil sealing the container. The graft contains a flexible area 9 that is capable of flexing as a function of the pressure difference between the inside and outside of the graft. In a normal stent graft the pressure outside the graft will be about 50 mmHg lower than the blood pressure inside the graft. Should a leak develop the pressure inside aneurysm 4 will increase and approach the blood pressure inside graft 2. Referring now to FIG. 2, the flexible area 9 will respond to this pressure difference by being pushed more into the graft when outside pressure increases. The natural shape of area 9, with no pressure present, is such that spikes 6 penetrate thin metal foil seal 11 and allow the chemical inside detector 5 to leak out. Spike 6 can be part of a thin and flexible clip 6′ which is also used to attache detector 5 to stent graft 2. Stent graft 2 is usually made from a combination of flexible wires 8 and a polymeric seal 7. Wires 8 are sometimes made of Nitinol. Graft 2 can be of the self-expandable type or can be expanded by a balloon or other means once inside the artery. The art of stent grafts and of percutaneous delivery is well known.

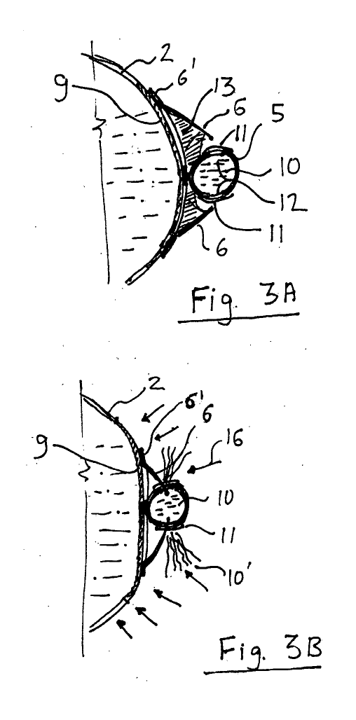

FIG. 3A shows a cross section of a stent graft incorporating the invention under normal conditions. The body of detector 5 forms a hermetically sealed container filled with chemical 10 attached to stent graft 2 by clip 6′. Chemical 10 is sealed inside detector 5 by two thin metal foil seals 11 that cover holes 12. Sharp spikes 6 are positions in proximity to foils 11 and will pierce them if clip 6′ is allowed to become flat. The natural shape of clip 6′ is flat; however the blood pressure inside graft 2 maintains the curved shape of clip 6′ as it is mounted on a flexible part of graft 2. A dissolvable material 13 protects seals 11 from barbs 6 during storage and deployment in the body. After deployment material 13 dissolves in the body and detector 5 is ready. The time it takes material 13 to dissolve depend on the material used. A standard dissolvable polymers used in the body are Polyglycolic Acid (PGA) and Polydioxanone (PDS). These are slow dissolving materials. Sugars such as glucose can be used as a rapidly dissolving protective material. In some cases it is desired to add a small non-dissolvable capsule of a material that has strong visco-elastic properties or a very high viscosity under spikes 6. Such a material, placed under or around spike 6 will prevent piercing seal 11 as a result of a sudden bump or shock, but will allow piercing in response to steady pressure. A suitable material is common pitch (also known as bitumen). It responds to a sudden shock it acts as a solid, but it flows under continuous pressure in a few minutes at body temperature. A drop of this material can be used as is, or sealed in a miniature flexible polymeric or metal bag, and placed under spike 6 to prevent accidental activation of detector 5. Alternatively, same material can also be used as a protective coating over foil seals 11. A transient pressure from spike 6 will not pierce the coating, while a constant pressure will. The response time can be modified by adding softeners such as waxes or oils to the pitch. Any material which has such a “slow flow” property, sometimes referred to as “cold flow”, can be used. Such behavior can also be achieved by forcing a liquid through a small orifice inside a sealed bellows, as used by devices known as “dashpots”.

FIG. 3B shows a cross section of a stent graft incorporating the invention under abnormal conditions. Blood leaking from an artery or stent graft causes increased pressure 16 outside graft 2. The reduced pressure differential across flexible area 9 allows clip 6′ to return to its natural flat state, causing spikes 6 to pierce foil seals 11 and releasing chemical 10 into the body, as shown by 10′.

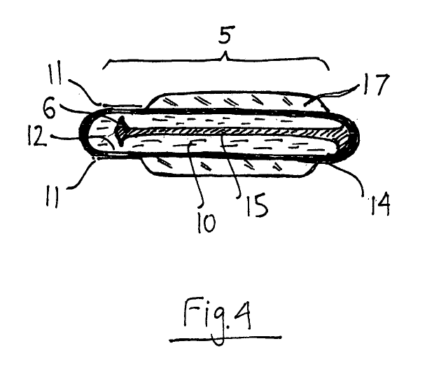

FIG. 4 shows another example of the invention, used to detect abnormal forces or distortion in the body. Such a detector can be used to detect abnormal bone fusion or pressure from an internal tumor. Detector 5 comprises of bendable sealed tube 14 containing chemical 10. Tube wall may include a bellows structure to make it more flexible and require smaller bending forces. A rigid central post 15 carries spikes 6 placed near openings 12 which are sealed by thin metal foils 11. When detector 5 is bent, spike 6 will pierce foil seal 11 and release chemical 10 into the body. If protection from transient forces is required, a high viscosity a visco-elastic material can be used. In this case it is preferred to make tube 14 double walled and fill the space with high viscosity material 17. The required viscosity for such materials is typically between one million cP to one billion cP. Foil seals 11 can be protected during storage and deployment by a coating of rigid dissolvable material, as explained in previous example. When central post 15 is replaced by a bimetallic element detector 5 becomes a temperature activated detector: when temperature exceeds a pre-set limit, the bending of post 15 will cause spike 6 to pierce foil seal 11. In this application no protection from transient forces is required. By placing a mechanical stop at the correct side of bimetallic post 15, detector can be triggered by a low temperature, a high temperature or both (when no stops are used).

It will be understood from the above examples that a detector for many other abnormal conditions can be made based on the same principle of releasing a chemical into the body. For example, a detector for pH level can be made by using a material which dissolves rapidly when pH is changes. Such a material can keep a detector as in FIG. 2 biased in one position and reverting to a second position (and releasing chemical) when material dissolves. Many materials are known that will not dissolve outside a certain pH range but will dissolve within that range.

For a stent graft application, by the way of example, detector 5 is made of type 0.2 mm thick 316L stainless steel in the form of a cylinder having a diameter of 5 mm and a length of 30 mm. Holes 12 are about 3 mm diameter. Foil seals 11 are is made of 2 um thick type 316L stainless foil, laser welded to detector body. Foils down to 1 um thick are available from special suppliers such as Lebow (www.lebowcompany.com). Such supplier can supply the thin foil already bonded to housing. In this case a separate filling hole is used, later sealed by laser or micro-TIG welding. Clip 6′ and spikes 6 are made from the same piece of work-hardened type 316L stainless steel about 0.1 mm thick. The tips of spike 6 should be very sharp. Another good choice for clip 6′ and spikes 6 is Nitinol, because of high elasticity and relatively high hardness. In this case thickness should be increased to about 0.2 mm.

The size of the detector is ultimately determined by the amount of chemical required to cause the desired reaction in the patient. The disclosed mechanism can be scaled to any size, from below 1 mm to over 10 mm in diameter. The detector can be used inside or outside lumens in the body.

The released chemical can be selected from a large group of candidates according to the desired effect on the patient, but in general there are four main groups:

Dyes, such as Methylene Blue or Sudan Black, causing visible change in skin or urine color.

Chemicals causing an unusual sensation. Foe example, dimethyl sulfoxide (DMSO) causes a strong garlic smell and taste in the patient.

Therapeutic agents. The above materials can be mixed with a drug acting as a “first aid” to the detected condition.

Specialized viruses and biological agents. The advantage of this group is that the detectors can be highly miniaturized, as the released agent multiplies in the body and causes a mild reaction identified by the patient. Viruses are desirable as they can be preserved for a long time but other biological agents can be used.

In some cases, when the application limits the size of the detector and the volume of the released chemical, sensitivity to the released chemical can be increased by external means. For example, a wrist band containing a sensitive chemical test for the chemical released in the body will allow the implanted detector to be reduced significantly. An external chemical test based on a wrist band containing chemicals (typically soaked and dried) can detect a specific chemical down to parts per million levels. This allows the amount of the released chemical to be in milligram range. External detecting aids based on electro-optical properties such as fluorescence can detect down to parts per billion, but require a power source and are more costly.

What is claimed is:

1. A device for implantation in a person, said device releasing a chemical in response to an abnormal condition, said chemical being detectable by the person.

2. A device as in claim 1 wherein the abnormal condition is an increase in pressure.

3. A device as in claim 1 wherein the abnormal condition is an increase in force.

4. A device as in claim 1 wherein the abnormal condition is an increase in temperature.

5. A device as in claim 1 wherein the abnormal condition is a change in pH.

6. A device as in claim 1 wherein the chemical is a dye.

7. A device as in claim 1 wherein the chemical is a dye changing the color of the person's urine.

8. A device as in claim 1 wherein the chemical is a dye changing the color of the person's skin.

9. A device as in claim 1 wherein the chemical is detectable by at least one of the senses of taste, smell, touch, eyesight and hearing.

10. A device as in claim 1 wherein the chemical is dimethyl sulfoxide.

11. A device as in claim 1 wherein the chemical is biological agent.

13. A device as in claim 1 wherein the chemical is a drug.

14. A device as in claim 1 wherein said detection is aided by an external detector.

15. A device as in claim 1 wherein said device does not respond to transient conditions.

16. A device as in claim 1 wherein said device is enabled only after said implantation.

17. A device as in claim 1 wherein said device contains a polymer dissolvable in the body.

18. A device as in claim 1 wherein said chemical is released by piercing a thin metal foil.

19. A device as in claim 1 wherein said device in made of metal.

20. A stent graft incorporating a system for the release of a chemical inside the body in case of abnormal conditions

21. An endovascular stent graft incorporating a system for the release of a chemical when pressure outside said graft increases beyond a pre-set limit.

22. An endovascular stent graft as in claim 21 wherein said pressure has to be present for at least a pre-set amount of time before said chemical is released.

23. An endovascular stent graft as in claim 21 wherein said system is prevented from operating prior to implantation in the body by a dissolvable polymer.

24. An endovascular stent graft as in claim 21 wherein said stent graft is delivered percutaneously.

25. An endovascular stent graft as in claim 21 wherein said chemical is detectable by at least one of the senses of taste, smell, touch, eyesight and hearing.
